(19) United States
(12) Patent Application Publication
(10) Pub. No.: US 2010/0267630 A1
(43) Pub. Date: Oct. 21, 2010

(54) USE OF LEPTIN IN THE PREVENTION OF UNHEALTHY FOOD HABITS AND CARDIOVASCULAR DISEASES

(75) Inventors: Andreu Palou Oliver, Palma De Mallorca (ES); Catalina Picó Segura, Palma De Mallorca (ES); Paula Oliver Vara, Palma De Mallorca (ES); Teresa Priego Cuadra, Palma De Mallorca (ES); Juana Sánchez Roig, Palma De Mallorca (ES); Mariona Palou March, Palma De Mallorca (ES); Aixa Tobaruela Arbona, Palma De Mallorca (ES)

Correspondence Address: MOORE & VAN ALLEN PLLC, P.O. BOX 13706, Research Triangle Park, NC, 27709, US

(73) Assignee: UNIVERSITAT DE LES ILLES BALEARS, Palma De Mallorca (ES)

(21) Appl. No.: 12/664,491

(22) PCT Filed: Jun. 20, 2008
(86) PCT No.: PCT/EP2008/057844

§ 371 Date: Jun. 11, 2010

(30) Foreign Application Priority Data
Jun. 20, 2007 (EP) 07380180.5

(51) Int. Cl. A61K 38/21; A61P 3/06; A61P 1/16

(52) U.S. Cl. 514/7.4

(57) ABSTRACT

The present invention is addressed to the use of leptin, a fragment of leptin or a mimetic product of leptin action, in the preparation of a pharmaceutical composition, a food composition and/or a nutritional supplement for the prevention of unhealthy food habits and/or cardiovascular disorders during adulthood.

### FIELD OF THE INVENTION

The present invention is related with the prevention of unhealthy food habits. In particular, it refers to the regulation of food preferences by carbohydrate-rich foods instead of fat-rich foods, as well as the improvement of appetite control regulation and other disorders as a result of an improvement of the leptin response. The improvement of food preferences that can be attained by the administration of the leptin-containing food results in a reduction of risk of cardiovascular disease and other disorders.

### BACKGROUND OF THE INVENTION

In general, the changes in lifestyle in developed countries, particularly a higher intake of fat associated to lower physical activity, have been considered one of the main causes of the rise in the incidence of metabolic disorders. The consumption of a diet high in fat is related to the development of cardiovascular diseases, the major cause of death in developed societies.

Nutrition is coming to the fore as a major modifiable determinant of chronic diseases, with scientific evidence increasingly supporting the view that alterations in diet have strong effects on health throughout life. Diet not only influences present health, but may determine whether or not an individual will develop diseases, as cardiovascular disease, much later in life (WHO, 2003: Diet, Nutrition and the Prevention of Chronic Diseases. Report of a Joint WHO/FAO Expert Consultation. WHO Technical Report Series no. 916. World Health Organization, Geneva). The World Health Organization (WHO) currently attributes one-third of all global deaths to cardiovascular diseases.

The relationship between dietary fats and cardiovascular diseases, especially coronary heart disease, has been extensively investigated, with strong and consistent associations emerging from a wide body of evidence accrued from animal experiments, as well as observational studies, clinical trials and metabolic studies conducted in diverse human populations (Kris-Etherton P. M. et al.: Summary of the scientific conference on dietary fatty acids and cardiovascular health: conference summary from the nutrition committee of the American Heart Association. Circulation, 2001, 103:1034-1039).

It is well known that the intake of saturated fatty acids raise total and low-density lipoprotein (LDL) cholesterol levels in blood, that are a risk factor for the development of cardiovascular diseases (Mensink R P, Katan M B. Effect of dietary fatty acids on serum lipids and lipoproteins. A meta-analysis of 27 trials. Arteriosclerosis and Thrombosis, 1992, 12:911-919) (Hu F B et al. Dietary fat intake and the risk of coronary heart disease in women. New England Journal of Medicine, 1997, 337:1491-1499).

According to the World Health Organization (WHO) (WHO, 2003), there are three critical aspects that have an impact on chronic diseases: (i) the development of risk factors during this period; (ii) the tracking of risk factors throughout life; and, in terms of prevention, (iii) the development of healthy or unhealthy habits that tend to stay throughout life, for example an excess of appetence for fat, and particularly for saturated fat rich foods. However, until the present invention, no specific nutritional factor or food component has been described that acts during development to determine the establishment of certain food preferences by carbohydrate-rich foods instead of fat-rich foods, thus altering food habits and/or the predisposition to other related disorders.

Dietary intake of fats strongly influences the risk of cardiovascular diseases such as coronary heart disease and stroke, through effects on blood lipids, thrombosis, blood pressure, arterial (endothelial) function, arrythmogenesis and inflammation. However, the qualitative composition of fats in the diet has a significant role to play in modifying this risk. According to the WHO the evidence shows that intake of saturated fatty acids is directly related to cardiovascular risk (WHO, 2003).

As a consequence, the traditional target is to restrict the intake of total fat to less than 30-35% of the energy intake and to restrict the intake of saturated fatty acids to less than 10%, of daily energy intake and less than 7% for high-risk groups. (WHO, 2003) (Eurodiet-report 2001: Nutrition and Diet for Healthy Lifestyles in Europe: the EURODIET evidence. Special Issue editors A. Ferro Luzzi, M. Gibney and M. Sjöström. http://eurodiet.med.uoc.gr/EurodietCoreReport.pdf. Public Health Nutrition 4 (2A&2B)).

Therefore, the modulation of food behaviour, including both control of appetite for some food compositions, and food preferences in favour of less fatty foods or with a lower caloric content, can provide a mechanism for the prevention of the development of metabolic disorders including cardiovascular diseases (Langley-Evans et al., Matern Child Nutr., 1, 142-148, 2005), particularly when food with a high caloric density or rich in fat, particularly saturated fat, is widely available, as happens in our developed societies. Other mechanisms, different from the control of appetite or food preferences, genetic or acquired, may be responsible for a higher or lower predisposition for the development, for instance, of diabetes and related disorders.

One of the more important signals playing a part in the maintenance of the energy balance and so of body weight is leptin, a circulating protein codified by the ob gene which is mainly expressed in the adipose tissue. Leptin plays a central role in the regulation of energy balance, inhibiting food intake and increasing energy waste (Zhang et al., Nature, 372, 425-432, 1994). This protein circulates in blood in a concentration that is quite proportional to the size of the fat depots; it passes through the haematoencephalic barrier by means of a saturable system, and exerts most of its effects on energy balance at a central level, afterwards the interaction of the protein with receptors located in hypothalamic neurons and in other regions of the brain (Tartaglia et al., Cell. 83, 1263-1271, 1995).

Animals with defects in the leptin route, because they do not produce the functional protein or because they express defective forms of its receptor, are characterised by hyperphagia and massive obesity of early appearance, as well as by suffering diabetes, hypothermia and infertility. In humans, congenic defects in the route of leptin (lack of leptin or of its receptor) are also related to morbid obesity of early appearance (Clement et al., Nature, 392, 398-401, 1998; Montague et al., Nature, 387, 903-908, 1997; Strobel et al., Nat. Genet., 18, 213-215, 1998). In this sense, the use of leptin in the treatment or prevention of diabetes mellitus (WO97/02004) whose direct cause is obesity was proposed.

Nevertheless, although it was thought that the short-term anorexigenic role of leptin could contribute to controlling the problem of obesity and related disorders in obese people, unfortunately, leptin administration has not been proven to be effective in the vast majority of cases of obesity in humans, due to the fact that these show resistance to the action of this protein.

It is known that, with age, the circulating levels of leptin increase (Li H. Matheny et al., Diabetes 1997, 46, 2035-9; Iossa S. et al., J Nutr. 1999, 129, 1593-6) and there is an impairment in sensitivity to this hormone (Qian H. et al., Proc Soc Exp Biol Med 1998, 219, 160-5; Scarpace P J. et al., Neuropharmacology, 2000, 39, 1872-9). Moreover, high levels of circulating leptin may favour the development of resistance to the anorexigenic effects of this hormone, which leads to perpetuating the development and maintenance of obesity and/or its complications. In fact, there is evidence suggesting that, in rats, leptin resistance would be the main determinant of body weight increase and age-related adiposity [Iossa, S. et al., J. Nutr., 1999, 129, 1593-6]. However, although the concentration of circulating leptin is usually considered to be proportional to body fat mass and this mass usually increases as we grow old, there is evidence that the increase in leptinemia and the development of leptin resistance with age occurs, at least in part, independently of the increase in adiposity (Gabriely I. et al., Diabetes, 2002, 51, 1016-21).

High leptin circulating levels have been also associated in humans with an increase in the risk of cardiovascular disease [Ren, J., J. Endocrinol., 2004, 181, 1-10] and development of insulin resistance [Huang, K C, et al., Int. J. Obes. Relat. Metab. Disord., 2004, 28, 470-5], and this even independently of body mass index/adiposity.

Although the adipose tissue is the main source, leptin is also produced by other tissues, including the placenta, stomach and mammary epithelium, and it is also present in maternal milk. Several studies show that breastfeeding, in comparison with bottle-feeding (infant formula milks), is related with a lower risk of suffering different complications in adulthood (Armstrong and Reilly, Lancet. 359, 2003-2004, 2002; Gillman et al., Jama., 285, 2461-2567, 2001; Kramer, J. Pediatr., 98, 883-887, 1981; von Kries et al., Bmj., 319, 147-150, 1999). Maternal milk has a lot of hormones and bioactive peptides that may play an important role in the development of the neonate, and could be responsible for exerting these effects of “metabolic imprinting” during this critical period of development. The fact that leptin is present in maternal milk, together with the results in rats and humans (Miralles et al., Obesity, 14, 1371-1377, 2006; Pico et al., Int. J. Obes., 2007), point to leptin as one of the bioactive compounds that could be responsible for the beneficial effects of breastfeeding and the lower medium/long-term incidence in the prevention of overweight and obesity.

However, to date no effect has been demonstrated of the intake of additional leptin during breastfeeding or during development, on the regulation or modulation of food habits in later stages of life or, in particular, in adulthood, as would be the case of a better appetite control or preference for less fatty foods, and, related to this, a higher sensitivity to leptin (and/or chronically maintained leptinemia within normal levels and lower to those that has been associated with risk for cardiovascular disease), which would considerably decrease the risk of suffering, in the short- and long-term, other metabolic and medical complications, such as cardiovascular diseases, and other illnesses or disorders related with the impairment of leptin sensitivity or effectiveness.

### BRIEF DESCRIPTION OF THE INVENTION

The authors of this invention have found that the administration of additional leptin to that acquired through maternal milk, in breastfeeding newly born mammals, makes it possible to modulate food habits towards a healthier diet by improving leptin sensitivity during adulthood. In particular, the administration of leptin during lactation has been demonstrated to allow the regulation of both appetite and food preferences in subsequent ages, decreasing the tendency to consume fat-rich and high energy foods, and promoting, instead, the consumption, among others, of carbohydrates. In addition, it also has been shown that leptin supplementation during lactation reduces the content of lipids on liver during adulthood, and the levels of triacylglycerides and of leptin in serum. These facts will significantly contribute to protecting from the subsequent development of metabolic and medical complications of the utmost importance, in particular cardiovascular diseases, that are the major contributor to the global burden of disease among the non-communicable diseases.

This new application of leptin is based on research carried out by the inventors on experimental animals, in which (at different ages and in adult life) they found an appetite reduction, associated with an improvement in the sensitivity to leptin and to a reduction in leptin levels (leptinemia), as well as a reduction in the preference for the intake of fat-rich foods and an increase in the intake of carbohydrates in adulthood, when these animals had been previously treated with an additional amount of leptin during their lactation. Other experiments have pointed out other benefits derived from the administration of leptin during lactation in relation to reduction of risk factors of cardiovascular disease, as a reduction of the levels of triglycerides and lipids in blood and liver in adulthood.

The observed effect of reduction of leptinemia and the maintenance or non-deterioration of leptin sensitivity in maturity, brought about by oral supplementation with leptin during lactation, are also interesting effects from a clinical point of view.

Therefore, an aspect of the present invention is related with the use of leptin, a fragment of leptin or a mimetic product of leptin action in the preparation of pharmaceutical composition for the prevention of unhealthy food habits and/or cardiovascular disorders during adulthood.

In another aspect, the present invention refers to the use of leptin, a fragment of leptin or a mimetic product of leptin action, in the preparation of a food composition or a nutritional supplement, for the prevention of unhealthy food habits and/or cardiovascular disorders during adulthood.

In a particular embodiment, the cardiovascular disease is selected from aneurysm, angina pectoris, Prinzmetal angina (spasm), acute coronary syndrome, arrhythmia, atherosclerosis, cardiac insufficiency, cardiomyopathy, cerebrovascular accident (stroke), cerebrovascular disease, congenital heart disease, congestive heart failure, myocarditis, valve disease, coronary artery disease, coronary heart disease, dilated cardiomyopathy, diastolic dysfunction, endocarditis, high blood pressure (hypertension), hypertrophic cardiomyopathy, mitral valve prolapse, myocardial infarction (heart attack), peripheral artery occlusive disease, restenosis and venous thromboembolism.

In another particular embodiment, the prevention of cardiovascular diseases can be caused as a consequence of the prevention of unhealthy food habits during adulthood. These unhealthy food habits mainly refer to unhealthy food preferences, such as the intake of fat-rich and high-energy food, to an excess of appetite or to a reduced frequency in the intake of meals per day.

The unhealthy food habits can, in turn, be caused by a deterioration of the leptin sensitivity, by a deficient action of endogenous leptin or by alterations accompanied by a deficient sensitivity or response to the administration of exogenous leptin, in subsequent ages or in adulthood.

In another particular embodiment, the prevention of cardiovascular diseases can also be caused by a sustained increase in leptinemia (elevated blood levels of leptin), which is increasingly seen associated with high risk of cardiovascular disease.

In another particular embodiment, the prevention of cardiovascular diseases can also be caused as a consequence of the prevention of increase of risk markers of cardiovascular diseases as blood and liver levels of triglycerides and total lipids in life stages following lactation and during adulthood.

In a particular embodiment, leptin, a fragment of leptin or a mimetic product of leptin action, is administered to breastfeeding newly born mammals or infant-formulae feeding humans (infants), more particularly to breastfeeding newly born humans or domestic animals.

In another particular embodiment, leptin, a fragment of leptin or a mimetic product of leptin action, is administered to nursing mothers and pregnant females, at higher doses than the ones planned for the breastfeeding mammals, with the purpose that, via the mother, sufficient leptin will arrive to foetuses or breastfeeding mammals.

Even in another particular embodiment, since there exist evidence of the presence of leptin receptor in the stomach and in the small intestine (Sobhani et al., Gut 2000, 47 (2), 178-183; Losato, FEBS Letters 2003, 423, 302-306; Barrenexe et al., Gut, 2002, 50, 797-802), leptin, a fragment of leptin or a mimetic product of leptin action, is administered to children and adolescents, with the aim of preventing an alteration in food habits and/or cardiovascular diseases during later stages of life.

Another aspect of the present invention relates to a method for the prevention of unhealthy food habits and/or cardiovascular diseases in adulthood, comprising the administration to a breastfeeding newly born mammal, nursing mothers, pregnant females, infants, children or adolescents, of an effective amount of leptin, a fragment of leptin or a mimetic product of leptin action.

A particular aspect of the present invention relates to a method for the prevention of unhealthy food habits and/or cardiovascular diseases in adulthood, comprising the administration to a breastfeeding newly born mammal, nursing mothers, pregnant females, infants, children or adolescents, of an effective amount of leptin, a fragment of leptin or a mimetic product of leptin action.

### BRIEF DESCRIPTION OF FIGURES

FIG. 1: Dietary preferences for a carbohydrate-rich diet (CR) or a fat-rich diet (FR) measured by the two-bottle test in 8-month-old male rats from control and leptin-treated during lactation group. Results are expressed as means ± SEM for the values of ingestion from 3 different days. * Statistical significance of the value of the intake of FR vs the value of the intake of CR (p<0.05, Student's t test).

FIG. 2: Serum leptin concentration under different feeding conditions (ad libitum, 14 hours fasting and 3 hours pair-fed refeeding after 14 hours fasting) in 9-month-old male Wistar rats which received a daily oral dose of leptin during lactation. Results are expressed as means ± SEM of 6 animals per group. T, effect of leptin treatment (p<0.05 two way ANOVA).

FIG. 3: Food intake after the leptin resistance test made in adult male Wistar rats that received a daily oral dose of leptin or vehicle during lactation. Food intake was measured after the intraperitoneal administration of leptin (2 mg/kg) (i.p leptin) or saline (vehicle) just before lights off at 20:00 h. Bars represent the mean value of the food intake during the 1^{st} and 2_{nd} hour after the intraperitonael leptin/vehicle administration (A), and the cumulative food intake 12 and 24 hours after the intraperitoneal leptin/vehicle administration (B). The Percentages of the reduction in food intake as effect of the intraperitoneal leptin administration respect to their controls injected with saline are indicated. * effect of intraperitoneal leptin administration (p<0.05, Student's t test).

FIG. 4: Serum levels of triacylglycerides (A) and hepatic lipid content (B) in 6-month-old male rats from control group and leptin-treated during lactation group, and fed after weaning with a normal fat (NF) or a high-fat (HF) diet. In A, results are expressed as mean values, and the percentage of decrease in serum triacylglycerides under NF and HF diet as effect of leptin treatment during lactation is indicated. In B, results are expressed as means ± SEM, and the percentage of decrease in the hepatic lipid content under NF and HF diet as effect of leptin treatment during lactation is indicated. * Statistical significance of the value under HF diet vs the value under NF diet (p<0.05, Student's t test). L, effect of leptin-treatment during lactation; D, effect of the type of diet (p<0.05, two way ANOVA).

### DETAILED DESCRIPTION OF THE INVENTION

In the context of the present invention, the following terms have the meaning detailed below:

It is understood by the term “leptin” the protein itself without modification, or rather, its definition may also include complete chain polypeptides of leptin which, in turn, may include modified molecules that contain adducts such as dextran, fatty acids or pegylated groups, or biologically active fragments or mimetic compounds of leptin action.

Complete chain polypeptides of leptin can be isolated from physiological sources or can be produced in a recombinant way. In a preferred embodiment of the present invention, leptin is a recombinant leptin that can come from different species, including humans, and it can be a whole or partially hydrolysed recombinant leptin. The term “partially hydrolysed leptin” refers to any fragment of this molecule that conserves the functional properties of leptin. The recombinant polypeptides may be generated in any expression system, such as yeast expression systems, bacterian from insects or mammals or other animals, and may be produced with or without their naturally-occurring secretion signal peptides. Adequate leptin fragments that can be used in the present invention include those described in U.S. Pat. No. 6,187,751, WO97/46585 and WO00/11173.

Likewise, different methods for cloning and purifying leptin compounds suitable for use in the present invention have been described in scientific literature [Pelleymounter et al., Science 269: 540-546 (1995); Campfield et al., Science 269: 546-549, (1995), and Chehab et al., Nat. Gen., 12: 318-320 (1996)].

For their part, the mimetic products of leptin action refer to those active agents that share one or more biological activities with leptin. They may be naturally-occurring polypeptides that share biological activities with leptin, such as the human obesity protein homolog-1 (WO01/25428), or the ciliary neurotrophic factor or axokine [Lambert et al., Proc. Natl. Acad. Sci., 98:4652-4657 (2001) and WO98/22128].

Alternatively, a mimetic product of leptin action may be a polypeptide of recombinant leptin which has replacements (including replacements by isomers, e.g.: one or more L-amino acids by the corresponding D-amino acids), eliminations and insertions of amino acids or chemical modifications of any amino acid relative to a native leptin sequence, which substantially retains or has a boosted leptin biological activity.

Also alternatively, a mimetic product of leptin action may be a drug based on a small synthetic molecule, a peptide or a polypeptide that is able to exert one or more of the biological effects of leptin; for example, a mimetic product of leptin is a drug or any other molecule having an spatial structure which is similar or coincides in part to that of any of the active sites of leptin. The active sites of leptin are those parts of the leptin molecule that are responsible for the effects described in the present application.

In a particular embodiment, natural or recombinant leptin or the fragments of leptin used in the present invention are derived from the same species as those that are going to be prevented. Therefore, humans will preferably be prevented with human leptin. However, leptin can be used to prevent species different from the ones the aforesaid comes from. In a preferred embodiment, leptin is obtained from humans, dogs, cats, mice, ferrets, apes, pigs or other bovine or sheep sources. Nevertheless, other forms of leptin may be used for the prevention of other species. For example, bovine, ovine, caprine, sow, buffalo, etc. leptin may be used to prevent humans.

The term “cardiovascular disease” refers to any disease that affects the cardiovascular system and that involve the heart or blood vessels (arteries and veins). In a particular embodiment, the cardiovascular disease is selected from aneurysm, angina pectoris, Prinzmetal angina (spasm), acute coronary syndrome, arrhythmia, atherosclerosis, cardiac insufficiency, cardiomyopathy, cerebrovascular accident (stroke), cerebrovascular disease, congenital heart disease, congestive heart failure, myocarditis, valve disease, coronary artery disease, coronary heart disease, dilated cardiomyopathy, diastolic dysfunction, endocarditis, high blood pressure (hypertension), hypertrophic cardiomyopathy, mitral valve prolapse, myocardial infarction (heart attack), peripheral artery occlusive disease, restenosis and venous thromboembolism.

In the field of the present invention, it is understood by “unhealthy food habits” those adverse habits that lead to the acquisition of unhealthy food preferences, in particular, a higher preference for fats, for saturated fatty acids or for foods that are a relevant source of these fats, and also to an excess of appetite.

Unhealthy food habits also include continuously maintained patterns of meal frequency of about 2-3 meals per day in humans, versus a frequency of meals of about 4-6 meals per day, which is considered more healthy. In humans, distributing eating in 5 or 6 times a day instead of the usually 2 or 3 is one of the typical recommendations of nutritionists. With only a period of 2 or 3 hours between meals, there is less feeling of being too hungry and less likely to splurge during next meal, a steady supply of glucose and other nutrients to body is achieved, thus preventing the high spikes and low drops in glucose and insulin levels.

For example, subjects consuming a single large daily meal has been shown to exhibit elevated fasting glucose levels and impaired morning glucose tolerance associated with a delayed insulin response during a 2-month diet period compared with those consuming three meals per day (Carlson et al.: Impact of reduced meal frequency without caloric restriction on glucose regulation in healthy, normal-weight middle-aged men and women. Metabolism 2007, 56(12):1729-34). Also, when meal frequency is decreased without a reduction in overall calorie intake, modest favourable changes occur in some cardiovascular disease risk factors, and hematologic variables (Stote et al.: A controlled trial of reduced meal frequency without caloric restriction in healthy, normal-weight, middle-aged adults. Am. J. Clin. Nutr. 2007October; 86 (4):1254-5). In general, epidemiological studies have shown that the consumption of smaller, more frequent meals, without increasing total food intake, results in an improvement in blood lipids and a reduction in the risk in cardiovascular disease in most patients (Bedelstein et al., Am. J. Clin. Nutr. 1992, 55, 664-9). Small, frequent meals are, in general, recommended for diabetics, as this assists with control of their blood sugar levels.

It is understood by “unhealthy food preferences” those preferences developed by animals or humans that cause the excessive consumption of hypercaloric and high-energy density foods, such as those with a high fat-content (mainly of animal source) and sugars, a high saturated fatty acids content or foods which are a source of these fats, potentially responsible for metabolic disorders such as obesity and diabetes.

It is understood by “excess of appetite” the development of a repetitive wish for food and/or drink that leads to an excessive food intake.

As mentioned above, the aforementioned unhealthy food habits can be caused as a consequence of deterioration in leptin sensitivity, of a deficient action of endogenous leptin or of alterations accompanied by a deficient sensitivity or response to exogenous leptin administration, during development and/or adulthood, and also in the suckling period. This deterioration in leptin sensitivity occurs with age and, to a certain extent, with body fat mass, associated to an increase in the circulating levels of this hormone. This decrease in sensitivity or response to leptin occurs both for endogenous leptin and for leptin administered in an exogenous way.

The term “deterioration of leptin sensitivity” refers to any decrease or loss of the capacity of the organism to produce a physiological response to leptin, that is, it leads to a deficient leptin action, whether it is endogenous-produced leptin or exogenous-administered leptin.

It is understood by “alterations that are accompanied by a deficient sensitivity to leptin” any condition or alteration of the organism that determines or brings with it a “deficient action of leptin” whether it be endogenous or exogenous, that is, a condition in which leptin effects (for example the appetite inhibitor effect or anorexigenic effect of leptin or other effects related with the control of appetite and energy waste, or effects on different functions related to reproduction, the immune system, hematopoyesis, angiogenesis, lipid metabolism, or other cellular functions related or not with the control of risk factors of cardiovascular diseases) are lower than the norm or those considered a referent.

Although adequate feeding and nutrition are important at all life stages, these are particularly important during childhood. Undesirable dietary habits can begin from birth or at a few months or years of life and can be established in a more or less persistent way in other stages, such as around puberty, with a tendency to be consolidated throughout the whole life. Childhood is, for this reason, a crucial period to act on food behaviour and on the mechanisms that determine its implementation, especially if this implementation lasts in posterior stages, because habits acquired at this stage are going to be determinant for the health of the future adult. By this reason, it is of particular relevance to prevent the appearance of these undesirable dietary habits form early ages, even from the lactating period and even, as far as possible, from a few months before birth. The biological plausibility and the interest of the new functions and applications of leptin have its basis on the presence of leptin in the first food eaten by humans (breast milk) and that this nutrient enters the digestive tract to achieve its function. However, this do dot precludes that other (non-oral) forms of administration can be also effective.

Thus, in a preferred embodiment of the present invention, the use of leptin is especially addressed at administration to breastfeeding newly born mammals, in a way that healthy habits in their feeding over a short- and long-term can be generated.

It is understood by “breastfeeding newly born mammal”, any mammal, and includes, although it is not limited to, domestic animals, rodents, primates and humans, in particular a human, in his/her first 6 months of life. Preferentially, this mammal is a human or a domestic animal, such as a dog or a cat, or a farm animal, such as a sheep or a cow. In a more preferred embodiment, the breastfeeding newly born mammal is a human being, male or female, of any race.

Leptin administration can be administered to a breastfeeding newly born mammal directly or through the mother's milk. For this reason, in another particular embodiment of the invention, the use of leptin, a fragment of leptin or a mimetic product of leptin action is administered to nursing mothers. In this way, it is possible to increase leptin concentration in maternal milk, with the breastfeeding mammal receiving an additional amount of this protein.

Likewise, leptin administration to a pregnant female mammal could favourably influence the metabolic pathways imposed to her descendant. Therefore, in another particular embodiment of the invention, the use of leptin, a fragment of leptin or of a mimetic product of leptin action is particularly administered to pregnant women in order to prevent her descendant from the development of alteration in food habits. In this aspect, leptin can be particularly effective when administered to women during the third trimester of pregnancy.

In addition, considering that leptin receptor is present in the stomach and intestine, leptin can also be administered to infants, children and adolescents.

It is understood by “infants” humans up to three-four years or infant-formulae feeding humans.

It is understood that any reference to humans (adults, infants or people at other ages) includes healthy humans, characterized by normal features (as normal parameters in blood, normal weight at birth, etc.), independently that also the application also refers to specific populations that can be predicted to be at more risk of developing alterations of food preferences, markers of cardiovascular diseases risk or risk to develop cardiovascular diseases.

In one particular embodiment of the invention, for its administration in the prevention of cardiovascular diseases, leptin is formulated in an appropriate pharmaceutical composition, in the therapeutically effective amount, together with one or more vehicles, adjuvants or pharmaceutically acceptable excipients.

In another particular embodiment, the pharmaceutical composition is administered orally, either in solid or liquid form. However, in the particular case in which leptin is administered to a breastfeeding mammal, the way of administration is in liquid form. Illustrative examples of pharmaceutical forms for oral administration include tablets, capsules, medicine in the form of granules, solutions, suspensions, etc. and may contain the conventional excipients, such as cohesives, diluents, disintegrators, lubricators, humectants, etc., and may be prepared by conventional methods.

In the particular case in which leptin is supplied to the nursing mother or to the pregnant female, the mode of administration may also be by other ways, such as parenteral or through patches.

In general, the therapeutically effective amount of leptin to administer will depend, among other factors, on the individual to be treated, on the presence of other agents with agonic or antagonic effects, on age, etc. For this reason, the doses mentioned in this invention must be considered only a guide for the expert in the subject, and this person must adjust the doses according to the aforementioned factors. However, leptin, a fragment of leptin or a mimetic product of leptin action can be administered one or more times a day, for example, 1, 2, 3 or 4 times a day, in a daily total typical amount comprising between 1 ng and 60 μg per day. In one particular embodiment, the total orally administered dose to a breastfeeding human or to an infant is approximately 1-2 μg per day during the first month of life, approximately 2-2.5 μg per day during the second month of life and approximately 2.5-3 μg per day from three to five months of life. In the particular case that leptin is supplied to the nursing mother or the pregnant female, the administered amounts could be considerably higher, especially between 0.1 μg and 60 mg, although they may vary depending on the conditions, type of leptin or mimetic and way of administration.

In another particular embodiment, for its administration in the prevention of cardiovascular diseases, leptin is formulated in an appropriate food composition or in a nutritional supplement, which comprises leptin, a fragment of leptin or a mimetic product of leptin action, and a food vehicle. To the effects of the present invention, it is understood for food vehicle any product capable of being used in human or animal feeding or that fits in the definition of food according to current European legislation. The choice of the suitable food vehicle for each case may be carried out by an expert in the subject from the conventional existing food vehicles in the state of the technique.

That food composition may be in the form of a soluble powder, it may be a concentrated liquid, a snack or a ready-to-use formulation suitable for oral intake or for enteral administration. Examples of these compositions may be, among others, a maternal milk, an infant formula milk for breastfeeding human or a continuation formula, a dairy product or a derivate such as a milk shake, milk in general (including full-cream milk, semi-skimmed, skimmed, concentrated, pasteurised, flavoured, fermented, soya milk, optionally supplemented with sugars, other carbohydrates, fat and other nutritional additives), a yoghurt, etc.; a juice; a flour product or derivate such as a cake, a bread, a cookie; an oil, sweets, such us chewing gum, candies, etc.

In the case that the food composition is milk, this may come from different mammal species, both humans and, for instance, farm animals and domestic animals. It is not necessary for leptin to come from the same species as the milk, but it is possible for instance to mix leptin from humans with milk from an animal or vice versa.

Typically, the way of mixing leptin and milk will depend on the way that this leptin is going to be administered and it will be selected so as to keep leptin activity in the final mixture. For example, leptin is generally sterilized by thermal treatment, as is the case of pasteurization. However, leptin activity can be seriously affected by this thermal treatment, and in this case it is convenient to add leptin after the sterilization process, or to use bacterial inactivating processes that do not inactivate leptin (radiation, high pressures, etc.).

The daily amount of leptin to administer by means of food composition in any of its forms will be comprised between 1 ng and 60 μg per day, although this amount can be exceeded depending on age and other conditions affecting or influencing the effectiveness of leptin. In one particular embodiment, the total administered dose to a breastfeeding mammal is approximately 1-2 μg per day during the first month of life, approximately 2-2.5 μg per day during the second month of life and approximately 2.5-3 μg per day from three to five months of life.

In a preferred embodiment of the present invention, this food composition is a children's food product. In an even more preferred embodiment, the children's product is powdered milk for breastfeeding neonates or liquid maternalized milk for breastfeeding neonates. In another preferred embodiment, this children's product is a paste or a baby food or a preparation of infant formula or of continuation. In these cases, the food composition contains leptin in a concentration between 0.1 and 30 μg/kg of product. Preferably, leptin concentration is 2.5 μg/kg of product.

In another preferred embodiment, the preparation for consumption is a nutritional supplement. This nutritional supplement may be a liquid composition for consumption, such as syrup or a drink, or a solid composition to consume, such as a tablet, capsule or powder to be reconstituted.

In another preferred embodiment, leptin, a fragment of leptin or a mimetic product of leptin action, is incorporated on the surface of a solid support, such as a dummy or object, so that, when sucking it, the product can be assimilated and incorporated in this way into the organism.

The amount of leptin in the food composition or in the nutritional supplement that may be ingested by a patient will depend on numerous factors such as the state of the patient, his/her body weight, age, among others. Nevertheless, the suitable amount will have to be prescribed by a specialist and will be adjusted in function of the previously mentioned variables. However, leptin, a fragment of leptin or a mimetic product of leptin action, may be administered in several doses, for example from 2 to 8 times a day, in order to administer the daily recommended amount or it may be ingested in one single dose.

On the other hand, leptin, a fragment of leptin of a mimetic product of leptin action, may be used together with other products or additional drugs that are useful in the prevention of cardiovascular diseases. These products or additional drugs may be part of the same pharmaceutical composition, food composition or nutritional supplement or, alternatively, may be provided in the form of a separate composition for simultaneous administration or not to the pharmaceutical composition, food composition or nutritional supplement that comprises leptin, a fragment of leptin or a mimetic product of leptin action.

The following example aims to illustrate the invention but it must not be considered as setting a limit in its scope.

### EXAMPLES

### General Procedure for the Selection of the Rat Groups to be Studied

Neonatal rats from different dams were selected. To obtain the pups, 3-month-old female virgin rats were matched with male rats. After matching, each female was placed in an individual cage with free access to water and standard chow (3000 Kcal/kg). Rats were kept in a room with controlled temperature (22° C.) and 12 h light-dark cycle. At day 1 after delivery 10 pups per dam were kept and randomly assigned into 2 groups: control group and leptin-treated group. From day 1 to day 20 of lactation, and during the first 2 h of the beginning of the light cycle, 20 μL, of the vehicle (water) or of murine leptin dissolved in water solution were daily and orally administered, using a pipette, to the control and leptin-treated group respectively. The amount of leptin given to animals was progressively increased from 1 ng of leptin on day 1 to 43.8 ng of leptin on day 20; this was calculated in other to supply five times the average amount of the daily leptin intake from maternal milk. On day 21, after weaning, both control and leptin-treated male rats were housed individually and fed on a standard chow diet.

### Example 1. Study of the Effect of Leptin Administration During Lactation on Food Preferences in Adulthood

A test [two bottles preference test (Kozak et al., Eur. J. Neurosci., 2005, 21, 2887-92)] was performed to assess the food preferences (carbohydrate-rich or fat-rich diet) in 8-month-old adult rats for the two selected groups as previously described: control rats and rats treated with leptin during the lactating period. This test consists of preparing two liquid meals, one carbohydrate-rich and the other fat-rich, both with the same caloric density (2.31 Kcal/g), and to determine the intake of each diet offered simultaneously for 1 hour.

It was observed that the total food intake consumed during 1 hour (considering both carbohydrate- and fat-rich diet) was similar in control and leptin-treated groups. However, both groups of animals differed in their dietary preferences. Control animals displayed a greater preference for the fat-rich diet than for the carbohydrate-rich diet (* p<0.05, Student's t test). On the other hand, leptin-treated rats showed a high preference for the carbohydrate-rich diet (FIG. 1).

### Example 2. Effect of the Supplementation of Leptin During Lactation on Leptinemia and Leptin Sensitivity in Adulthood.

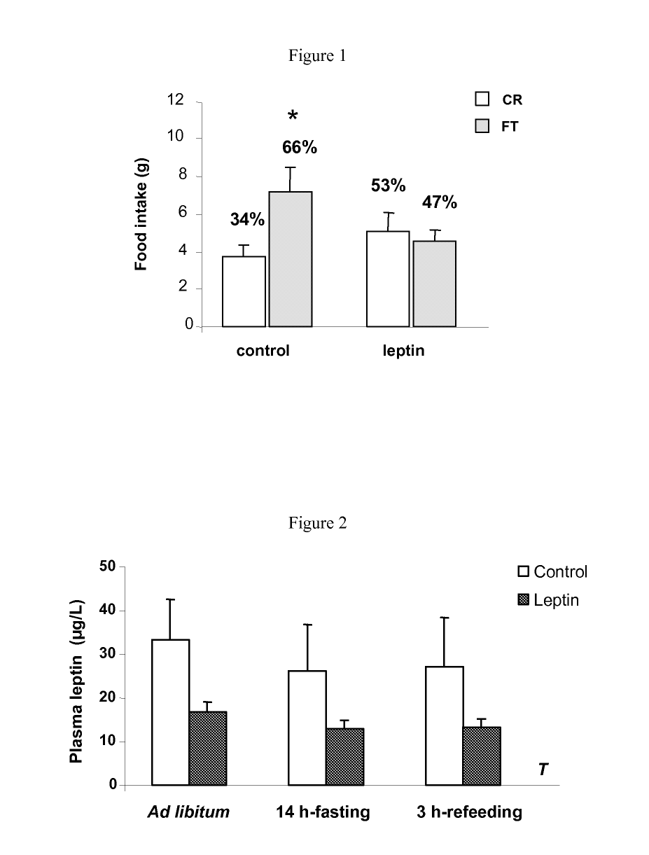

For this study, control and leptin-treated rats during lactation as described above were used. These rats, from both groups, were subject to 3 different feeding conditions: ad libitum, 14 hours fasting, and 3 hours refeeding after 14 h fasting and circulating leptin levels were analysed, afterwards. FIG. 2 shows that circulating leptin levels were significantly lower in rats that were treated with leptin during lactation than in their control counterparts.

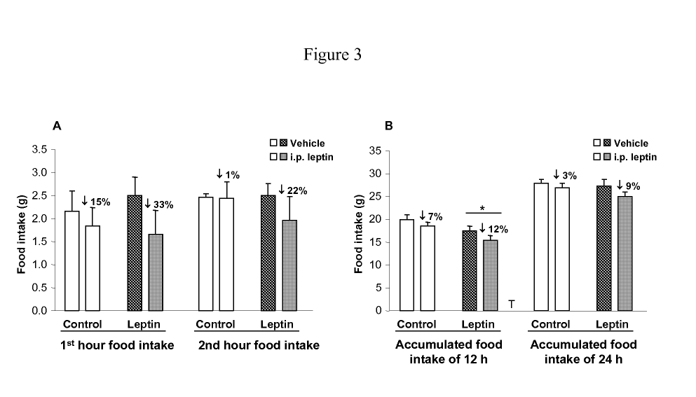

In addition, rats that were treated with leptin during lactation were more sensitive to the anorexigenic effects of leptin than control animals. This was proved by determining the effects of the intraperitoneal administration of exogenous leptin (compared to the intraperitoneal administration of saline) on food intake of control and leptin-treated rats. Intraperitoneal administration of leptin (2 mg/kg) resulted, after 1 h, in a slight, but not significant, decrease in food intake in both groups, which was more pronounced in leptin-treated than in control group (reduction of 23% and 15%, respectively compared to their control injected with saline) (FIG. 3). In addition, the anorexic effect of exogenous leptin was more persistent in leptin-treated than in control animals: during the second hour after the intraperitoneal leptin administration, the effect was imperceptible in control animals while it was still evident in leptin-treated rats which showed a 22% lower food intake in this period than their respective controls which received an intraperitoneal administration of saline. Considering the first 12 h after the intraperitoneal administration of leptin (corresponding to the dark period), the anorectic effect of exogenous leptin was significant in leptin-treated rats (p<0.05, Student's t test) but not in the control group. No significant effects of exogenous leptin on food intake in either of the two groups were observed when the period of 24 h after the exogenous administration of leptin was considered.

Therefore, the role of leptin supplied during lactation in the control of appetite in further ages and in another important aspect of the control of feeding behaviour in later life, such are food preferences, is demonstrated. The role of leptin supplied during lactation in reducing leptinemia and improving the sensitivity to this hormone is also demonstrated.

### Example 3. Effect of the Supplementation of Leptin During Lactation on Total Food Intake and on Food Intake Rhythms.

Cumulative food intake during the whole studied period (from day 21 to 15 months of age) was significantly lower in leptin-treated rats than in their controls (controls: 33317±474 kcal, leptin-treated group: 30827±761 kcal; p<0.05 Students's t test). In addition, both groups of animals followed different food intake rhythms. The average daily consumption of food was more distributed through the day in leptin-treated animals than in their controls: control animals consumed a mean of 71.9±3.0% of their total food intake during the dark period, and leptin-treated animals consumed 63.3±2.6 % of their food intake. This means that leptin-treated rats increased the frequency of meals without increasing the total amount of calories consumed during the day, which when applied to humans may have interest to improve dietary habits.

### Example 4. Effects of Leptin Supplementation During Lactation on Serum Levels of Triacylglycerides and on the Liver Content of Lipids in Adulthood.

For this study, neonate rats were orally treated with physiological doses of leptin during lactation, as described in the general procedure. On day 21, after weaning, both control and leptin-treated male rats were housed individually and were fed with a standard chow diet (normal fat; NF) diet (containing 10% calories from fat) or a high fat (HF) diet (containing 45% calories from fat). At the age of 6 months, animals were killed by decapitation under fed conditions, during the first 2 h of the beginning of the light cycle. The liver was rapidly removed and frozen in liquid nitrogen. Blood was collected, stored at room temperature for 1 h and overnight at 4° C., and was then centrifuged at 1000 g for 10 min to collect the serum, which was stores at −20° C. The lipid content in the liver was measured by the Folch method (Folch et al., J. Biol. Chem., 1957, 226, 497-509). Triacylglyceride levels in serum were measured enzymatically using a commercial kit (Sigma, Madrid, Spain).

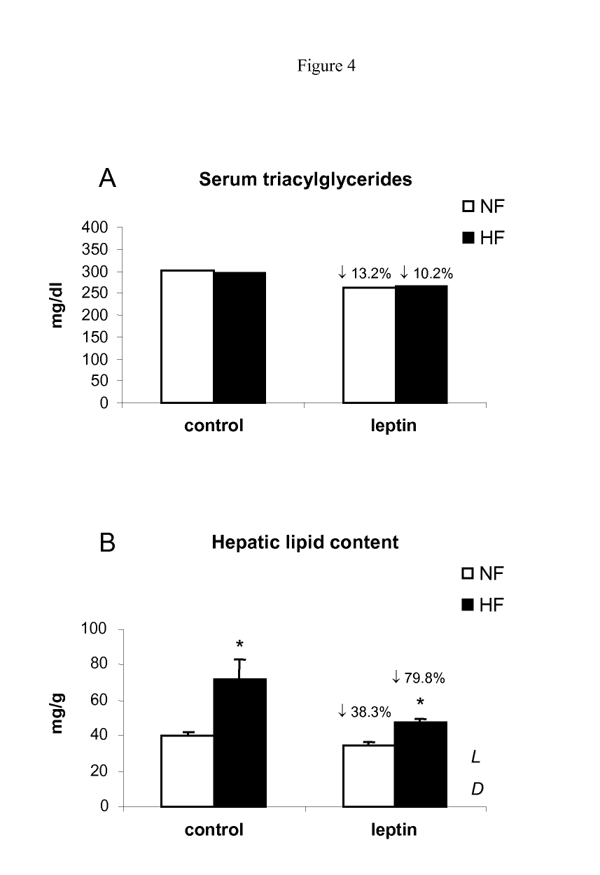

Results showed that serum triacylglyceride levels in 6-month-old animals were lower in leptin-treated animals; both under NF (decrease of 13.2%) and HF (decrease of 10.2%) diet (FIG. 4A). The benefits of the supplementation with leptin during lactation on tissue lipid content were more evident in liver. Total lipid content in liver was significantly lower in leptin-treated animals, both in animals under NF (decrease of 14.4%) and HF (decrease of 34.2%) diet (p<0.05, two way ANOVA) (FIG. 4B). Of interest, HF diet feeding resulted in an increase in the hepatic lipid content in both control and leptin-treated animals, but the increase was much lower in leptin-treated animals (38.3%) compared with the increase observed in untreated controls (79.8%).

1.-18. (canceled)

19. A method of lowering liver lipid content in an adult mammal, said method comprising:
administering a supplemental amount of leptin to a neonate mammal during lactation of said neonate mammal,
wherein the administration of leptin to the neonate mammal results in a lower lipid content in the liver of the mammal at adulthood relative to an equivalent aged mammal that was not administered the supplemental amount of leptin.

20. The method of claim 19, wherein the administration is oral.

21. The method of claim 19, wherein the administration is daily.

22. The method of claim 19, wherein the mammal is a human.

23. The method of claim 19, wherein the amount of leptin administered is greater than the average amount of daily leptin intake from maternal milk of the mammal.

24. The method of claim 19, wherein the amount of leptin administered is about five times the average amount of daily leptin intake from material milk of the mammal.

25. The method of claim 19, wherein the leptin is administered to breastfeeding neonate humans.

26. The method of claim 19, wherein the leptin is administered in water, a dairy product or derivate thereof, a juice, a flour product or derivate thereof, an oil or a sweet.
